(19) United States
(12) Patent Application Publication
(10) Pub. No.: US 2025/0195068 A1
(43) Pub. Date: Jun. 19, 2025

(54) RELOAD COVER FOR SURGICAL STAPLING SYSTEM

(71) Applicant: Applied Medical Resources Corporation, Rancho Santa Margarita, CA (US)

(72) Inventors: Jeffrey Weidner, Rancho Santa Margarita, CA (US); Nicholas Humphreys, Aliso Viejo, CA (US); Timothy M. Hopkins, Rancho Santa Margarita, CA (US)

(21) Appl. No.: 19/070,171

(22) Filed: Mar. 4, 2025

Related U.S. Application Data
(63) Continuation of application No. 18/338,644, filed on Jun. 21, 2023, now Pat. No. 12,285,167.
Application No. 18/338,644 is a continuation of application No. 16/833,288, filed on Mar. 27, 2020, now Pat. No. 11,717,293.
(60) Provisional application No. 62/826,194, filed on Mar. 29, 2019.

(51) Int. Cl. A61B 17/072; A61B 17/00

(52) U.S. Cl. CPC A61B 17/07207; A61B 2017/00477; A61B 2017/0053; A61B 2017/07271

(57) ABSTRACT

Surgical stapler systems can include a jaw assembly with one jaw defining a reload support capable of receiving and firing multiple disposable reload cartridges in a single surgical procedure. The reload cartridge can be protected by a reload cover engageable therewith. The reload cover can include snap features configured to prevent manual removal of the reload cover from the reload cartridge before proper positioning and installation in the reload support. The reload cover can desirably maintain a plurality of staples within staple pockets in the reload cartridge and can maintain a staple deployment mechanism in an unfired position.

### CROSS-REFERENCE TO RELATED APPLICATIONS

This application is a continuation of U.S. patent application Ser. No. 18/338,644 entitled “Reload Cover for Surgical Stapling System” filed on Jun. 21, 2023 which is a continuation of U.S. patent application Ser. No. 16/833,288 entitled “Reload Cover for Surgical Stapling System” filed on Mar. 27, 2020, now issued U.S. Pat. No. 11,717,293, which claims priority to and benefit of U.S. Provisional Patent Application Ser. No. 62/826,194 entitled “Reload Cover for Surgical Stapling System” filed on Mar. 29, 2019, each of which is incorporated herein by reference in its entirety.

### BACKGROUND OF THE INVENTION

### FIELD OF THE INVENTION

The present application relates generally to surgical occlusion instruments and, more particularly, to surgical staplers having reloads with removable covers.

### DESCRIPTION OF THE RELATED ART

Surgical staplers are used to approximate or clamp tissue and to staple the clamped tissue together. As such, surgical staplers have mechanisms to ensure that tissue is properly positioned and captured and to drive staples through the tissue. As a result, this has produced, for example, multiple triggers and handles in conjunction with complex mechanisms to provide proper stapling of the clamped tissue. With these complex mechanisms, surgical staplers can have increased manufacturing burdens, as well as potential sources for device failure and confusion for the user. Thus, reliable stapling of clamped tissue without complex mechanisms is desired.

Surgical staplers can further include replaceable reload cartridges such that multiple cartridges can be used with a stapler in a single surgical procedure. Further improvements to the reload cartridges are desirable to facilitate proper placement of the initial and each subsequent reload cartridges for use in the surgical stapler.

### SUMMARY OF THE INVENTION

In certain embodiments, a surgical stapling device is provided herein. The surgical stapling device comprises an elongate shaft, a jaw assembly, a reload cartridge, and a reload cover. The jaw assembly comprises a reload support configured to receive the reload cartridge. The reload cover is releasably positionable on the reload cartridge. The reload cartridge comprises at least one protruding boss extending laterally outwardly therefrom. The reload cover comprises at least one snap feature extending therefrom, each snap feature engageable with a corresponding protruding boss. The at least one snap feature is not manually disengageable from the at least one protruding boss. Installation of the reload cartridge and reload cover to the reload support disengages the at least one snap feature from the at least one protruding boss.

In certain embodiments, a reload cartridge assembly for a surgical stapler is provided herein. The reload cartridge assembly comprises a cartridge body and a reload cover. The cartridge body has a generally elongate profile extending from a proximal end to a distal end. The cartridge body comprises an upper, tissue contacting surface and a pair of lateral surfaces. The upper, tissue contacting surface has a plurality of rows of staple pockets formed therein and at least one seating boss protruding upwards from the upper surface at the proximal end. The pair of lateral surfaces each comprise a protruding boss extending laterally outwardly at the distal end of the cartridge body. The reload cover is removably positionable on the upper surface. The reload cover has a generally planar configuration and a generally elongate profile extending from a proximal end to a distal end. The reload cover comprises a pair of latch arms extending downwardly from the distal end thereof. Each of the latch arms is engageable with a corresponding protruding boss to retain the reload cover to the upper surface of the cartridge body such that the reload cover is not manually removable.

In certain embodiments, a method of installing a reload cartridge to a reload support is provided herein. The surgical stapler comprises a jaw assembly comprising a reload support and the reload cartridge comprises a reload cover engaged thereto. The method comprises positioning a proximal end of the reload cartridge into a proximal end of the reload support. The method further comprises advancing a protruding boss adjacent the distal end of the reload cartridge into an engagement notch of the reload support such that the engagement notch releases a snap feature of the reload cover. The method further comprises removing the reload cover once the snap feature has been released.

### BRIEF DESCRIPTION OF THE DRAWINGS

FIG. 1 is a perspective view of an embodiment of surgical stapling device with the jaws in an open configuration;

FIG. 2 is a perspective view of an embodiment of a reload shaft for the surgical stapling device of FIG. 1 with the jaws in an open configuration;

FIG. 3 is a partially exploded perspective view of a jaw assembly, reload cartridge, and reload cover of the surgical stapling device of FIG. 1;

FIG. 4 is an end view of a reload cartridge and a reload cover of the surgical stapling device of FIG. 1;

FIG. 5 is a perspective view of the reload cartridge and reload cover of FIG. 4;

FIG. 6 is a cross-sectional end view of a jaw assembly with a partially installed reload cartridge and reload cover of the surgical stapling device of FIG. 1;

FIG. 7 is a perspective view of a reload cartridge of the surgical stapling device of FIG. 1;

FIG. 8 is a perspective view of a reload cover of the surgical stapling device of FIG. 1;

FIG. 9 is a top view of the reload cover of FIG. 8;

FIG. 10 is a bottom view of the reload cover of FIG. 8;

FIG. 11 is a side view of the reload cover of FIG. 8; and

FIG. 12 is a cross-sectional end view of the reload cover of FIG. 8.

### DETAILED DESCRIPTION OF THE INVENTION

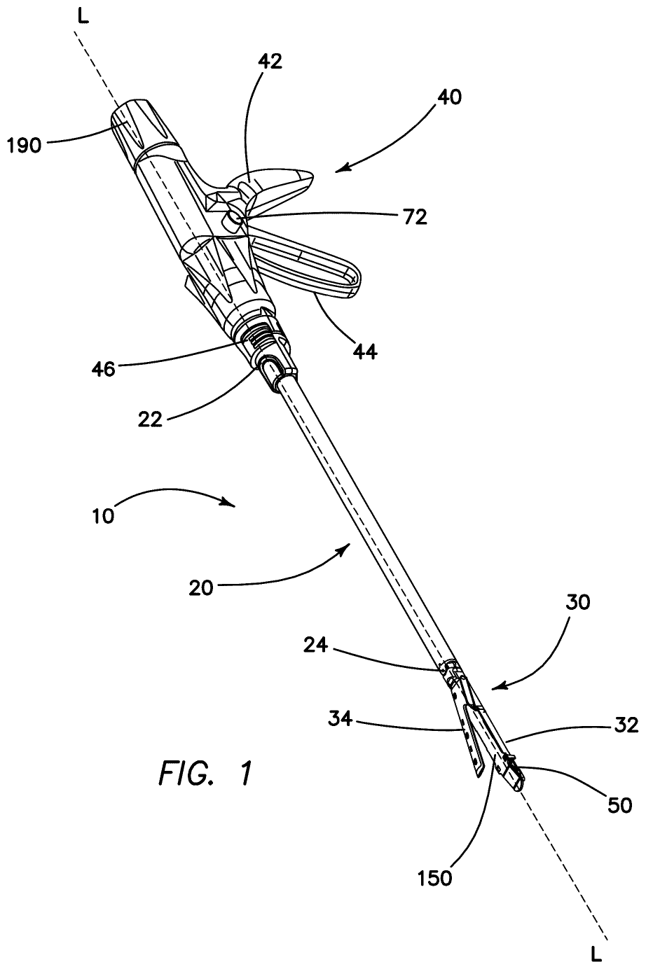

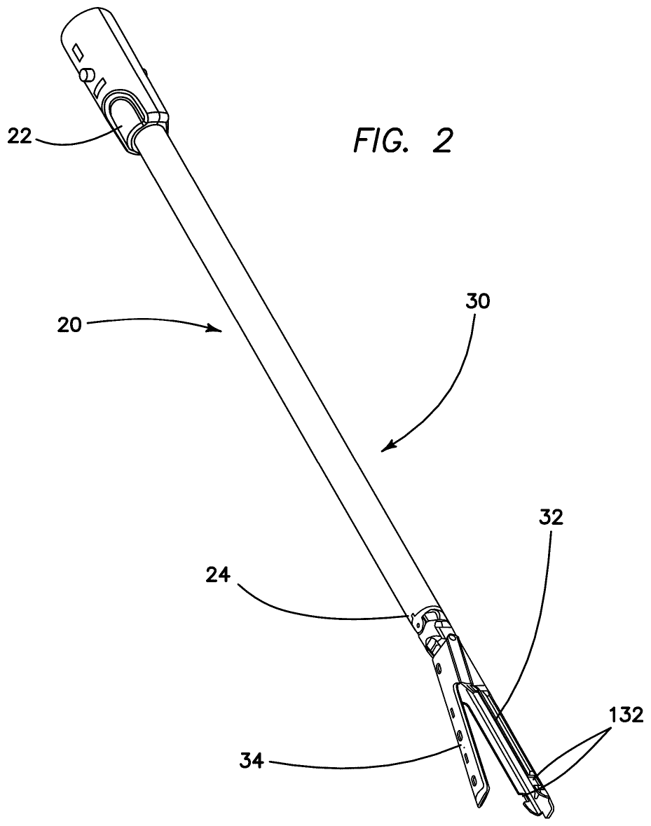

With reference to FIGS. 1-2, embodiments of surgical stapling device are illustrated. The embodiment of stapling device illustrated in FIGS. 1-3 includes a mechanical handle assembly 40, although various aspects of the reload cartridge and reload cover described herein can be used in conjunction with an electrically powered handle assembly. The illustrated embodiment of surgical stapler 10 comprises an elongate shaft 20, a jaw assembly 30, and a handle assembly 40. Various aspects of the elongate shaft 20 and jaw assembly 30 described herein can be used interchangeably with either the mechanical handle assembly 40 or a powered handle assembly. FIG. 1 illustrates the surgical stapler 10 with the jaw assembly 30 in an open configuration. FIG. 2 illustrates a removable reload shaft assembly comprising the elongate shaft 20 and jaw assembly 30 of the surgical stapler 10 with the jaw assembly 30 in an open configuration.

With continued reference to FIGS. 1 and 2, the illustrated embodiment of surgical stapler 10 can be sized and configured for use in laparoscopic surgical procedures. For example, the elongate shaft 20 and jaw assembly 30 can be sized and configured to be introduced into a surgical field through an access port or trocar cannula. In some embodiments, the elongate shaft 20 and jaw assembly 30 can be sized and configured to be inserted through a trocar cannula having a relatively small working channel diameter, such as, for example, less than 8 mm. In other embodiments, elongate shaft 20 and jaw assembly 30 can be sized and configured to be inserted through a trocar cannula having a larger working channel diameter, such as, for example, 10 mm, 11 mm, 12 mm, or 15 mm. In other embodiments, it is contemplated that certain aspects of the surgical staplers described herein can be incorporated into a surgical stapling device for use in open surgical procedures.

With continued reference to FIGS. 1 and 2, as illustrated, the elongate shaft 20 comprises a generally tubular member. The elongate shaft 20 extends from a proximal end 22 to a distal end 24. The elongate shaft 20 defines a central longitudinal axis, L. of the surgical stapler 10 extending between the proximal end 22 and the distal end 24. The jaw assembly 30 and reload cartridge 50 likewise have generally elongate configurations, each extending from respective a proximal end to a respective distal end, along the central longitudinal axis when aligned with the elongate shaft 20.

With continued reference to FIGS. 1 and 2, in the illustrated embodiment, the jaw assembly 30 is coupled to the elongate shaft 20 at the distal end 24 of the elongate shaft 20. The jaw assembly 30 comprises a first jaw 34 pivotally coupled to a second jaw 32. In the embodiment illustrated in FIGS. 1-2, the jaw assembly is articulably coupled to the elongate shaft such that the jaw assembly can be selectively positioned at an articulated position with respect to the central longitudinal axis L and an aligned position with respect to the central longitudinal axis L. The handle assembly of FIG. 1 includes an articulation knob 190 and articulation mechanism configured to provide continuously selectable articulation of a jaw assembly of an elongate shaft assembly through an articulation range. In an initial configuration, the second jaw 32 includes a plurality of staples positioned within a reload cartridge 50 positioned therein. Thus, the second jaw 32 defines a reload support.

With continued reference to FIGS. 1 and 2, in the illustrated embodiment, the jaw assembly 30 can be actuated from an open configuration (FIG. 1) to a closed configuration to a stapling configuration by an actuation member or beam that is longitudinally slidable within the elongate shaft. In an initial position, the beam can be positioned at the distal end 24 of the elongate shaft 20. With the beam in the initial position, the first jaw 34 is pivoted away from the second jaw 32 such that the jaw assembly 30 is in the open configuration. The second jaw 32 has an elongate configuration extending from the distal end of the elongate shaft. The actuation beam engages the first jaw 34 upon translation of the actuation member or beam distally along the longitudinal axis L. Translation of the actuation beam distally from the initial position a first distance can actuate the jaw assembly from the open configuration to the closed configuration. With the jaw assembly 30 in the closed configuration, the actuation beam can be returned proximally the first distance to return the jaw assembly 30 to the open configuration. A distal end of the actuation beam can advance a staple slider configured to deploy staples from the second jaw 32 such that further translation of the actuation beam distally past the first distance deploys the plurality of staples 36 from the second jaw 32.

With reference to FIGS. 1-3, in the illustrated embodiment, the handle assembly is coupled to the elongate shaft 20 at the proximal end 22 of the elongate shaft 20. As illustrated, the handle assembly 40 has a pistol grip configuration with a housing defining a stationary handle 42 and a movable handle 44 or trigger pivotably coupled to the stationary handle 42. It is contemplated that in other embodiments, surgical stapler devices including aspects described herein can have handle assemblies with other configurations such as, for example, scissors-grip configurations, or in-line configurations. The handle assembly 40 houses an actuation mechanism configured to selectively advance an actuation shaft responsive to movement of the movable handle 44 to actuate the actuation beam within the elongate a shaft a first distance in an open-close stroke to close the jaw assembly from an initial open position, a second distance beyond the first distance in a firing stroke to fire staples, and to return the actuation beam the second distance and the first distance to an initial position. In certain embodiments, a sliding selector 72 on the handle assembly can allow a user to select whether the handle assembly operates to actuate a jaw assembly in an open-close stroke or a firing stroke. Various embodiments of handle assemblies and associated actuation mechanisms are disclosed in U.S. Pat. No. 9,668,732, entitled “Surgical Stapler Handle Assembly Having Actuation Mechanism With Longitudinally Rotatable Shaft” and U.S. Patent application Ser. No. 15/485,620, filed Apr. 12, 2017, entitled “Surgical Stapler Having Articulation Mechanism,” both of which are incorporated by reference herein in their entireties.

With continued reference to FIGS. 1-3, in some embodiments, the surgical stapler 10 can include the plurality of staples positioned in a disposable reload cartridge 50 while the handle assembly 40 and elongate shaft 20 are configured to be reused with multiple staple reload cartridges in a surgical procedure. The surgical stapler can include one or more grasping and firing lockout mechanisms that can limit functionality of the handle assembly to alert a user and enhance patient safety if no reload cartridge is present in the jaw assembly or if a partially or fully fired reload cartridge is present in the jaw assembly. In certain embodiments, a staple deployment member, such as a translatable sled or slider within the reload cartridge 50 can defeat one or more lockout mechanisms when the staple deployment member is in a proximal position in the jaw assembly, corresponding to an unfired reload cartridge is present in the surgical stapler 10.

With reference to FIG. 1, the handle assembly 40 includes a coupler 46 at the distal end thereof. The coupler 46 is adapted to engage the elongate shaft 20 of the surgical stapler 10. The coupler 46 can have a bayonet connection having an outer connector that can removably couple the handle assembly 40 to the elongate shaft 20, and an inner connector that can removably couple the actuation shaft of the handle assembly 42 to the actuation member of the elongate shaft 20. Accordingly, the surgical stapler 10 can be configured such that the handle assembly 40 can be reused with multiple disposable shafts and/or reload cartridges during a surgical procedure. It is contemplated that in other embodiments, the handle assembly and some portion of the elongate shaft can be reusable while a remainder of the elongate shaft and the jaw assembly define a disposable cartridge. In certain other embodiments, the handle assembly and the elongate shaft can be reusable while the jaw assembly defines a disposable cartridge. In still other embodiments, a jaw insert housing a plurality of staples can define a disposable cartridge while the remainder of the surgical stapler is reusable.

As noted above, the elongate shaft, jaw assembly, reload cartridge, and reload covers described herein can also be used in conjunction with a powered stapler handle assembly. Various embodiments of powered handle assemblies and associated actuation mechanisms are disclosed in U.S. patent application Ser. No. 15/486,227, filed Apr. 12, 2017, entitled “Reload Shaft Assembly for Surgical Stapler;” U.S. patent application Ser. No. 15/486,008, filed Apr. 12, 2017, entitled “Surgical Stapler Having a Powered Handle;” and U.S. patent application Ser. No. 16/287,748, filed Feb. 27, 2019, entitled “Surgical Stapler Having a Powered Handle;” all of which are incorporated by reference herein in their entireties.

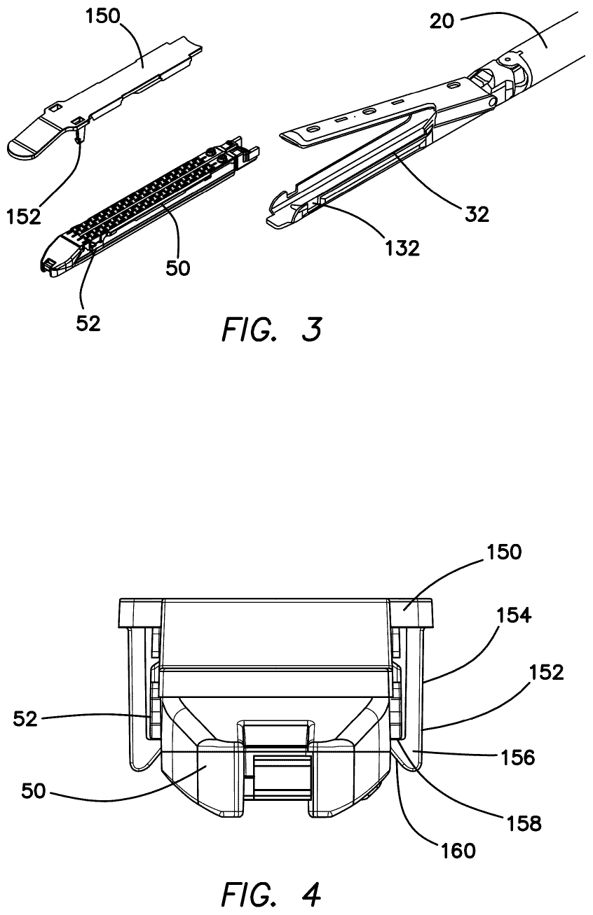

With reference to FIG. 3, a perspective view of the jaw assembly of the elongate shaft 20 is illustrated with the reload cartridge 50 and reload cover 150 removed from the second jaw 32. As illustrated, the reload cover 150 overlies the reload cartridge 50, and the reload cartridge 50 is positionable in the reload support defined by the second jaw 32. As further described herein, the reload cover 152 can include one or more snap features 152 engageable with a corresponding one or more protrusions or bosses 52 on the reload cartridge 50. The snap features 152 and bosses 52 are alignable with an engagement notch 132 or release recess formed in the second jaw 32 when the reload cartridge 50 is properly positioned and seated within the second jaw 32. In the illustrated embodiment, the reload cartridge 50 includes a plurality of staples disposed therein, each staple positioned in its own staple pocket formed through a body of the reload cartridge. In the illustrated embodiment, the staple pockets are arranged in a plurality of longitudinally extending rows. An upper surface of the reload cartridge 50 defines a tissue contact surface which, in certain embodiments, can be substantially planar. The reload cartridge further comprises a blade channel formed therein. As illustrated, the blade channel longitudinally extends between two adjacent rows of staple pockets such that translation of a cutting blade through the blade channel transects tissue between rows of staples that have been deployed into tissue positioned in the jaws when staples are fired.

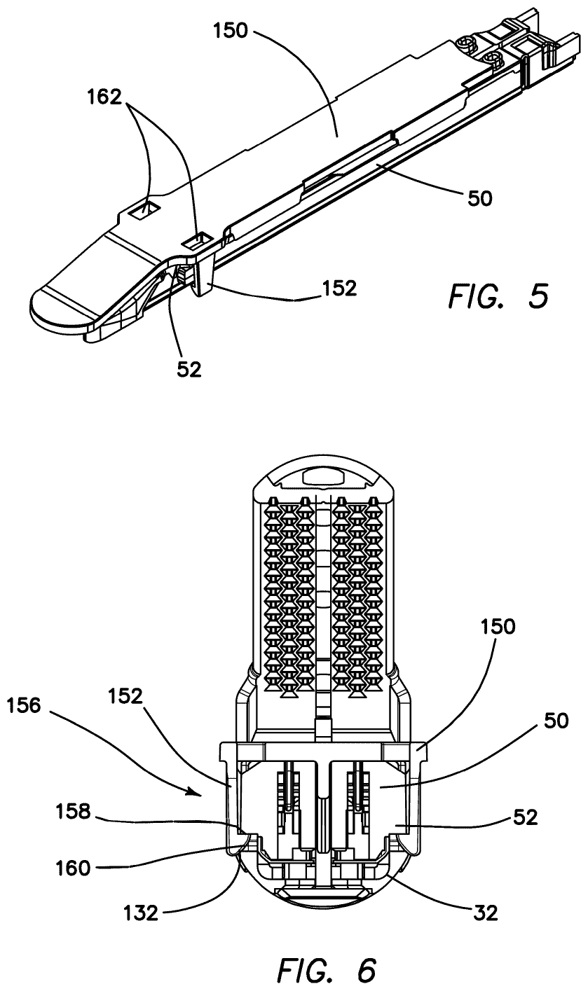

With reference to FIGS. 4-5, views of the reload cartridge 50 and reload cover 150 engaged with the reload cartridge 50 are illustrated. As illustrated, the reload cover 150 is engaged with the reload cartridge 50 to abut, contact, or be spaced adjacent to the upper surface of the reload cartridge. Thus, the reload cover 150 can protect the tissue contact surface of the reload 50 from impacts which could cause marring or damage during shipping and transport of the reload cartridge 50. Desirably, with the reload cover 150 positioned directly on the upper surface of the reload cartridge 50, the plurality of staples are retained within the staple pockets in the reload cartridge 50. Thus, prior to and during installation of the reload cartridge in the jaw assembly, the plurality of staples are unlikely to be inadvertently dislodged from their unfired positions by any impacts or other vibratory forces during shipment or manipulation of the reload cartridge.

With continued reference to FIGS. 4-5, it can be desirable that the reload cover 150 is maintained on the reload cartridge in a manner that is not easily manually removable such that a user would be largely unable to remove the reload cover before installation in the jaw assembly. Advantageously, such an arrangement can require a user to properly position and install the reload cartridge in the jaw assembly before the reload cover can be removed. In the illustrated embodiment, the reload cover 150 can comprise at least one snap feature 152 configured to engage a corresponding at least one boss 52 protruding from the reload cartridge. In the illustrated embodiment, the reload cover 150 comprises two snap features 152 laterally outwardly positioned adjacent a distal end of the reload cover 150. In other embodiments, it is contemplated that a reload cover can comprise more than two snap features and/or can comprise snap features positioned adjacent the proximal end of the reload cover. Each snap feature comprises an arm 154 extending downwardly from a generally planar cover surface of the reload cover. The arm 154 comprises a latching tooth 156 positioned at an end thereof opposite the cover surface of the reload cover. The snap features 152 are flexible such that upon initial assembly with a reload cartridge, the snap features will flex laterally outwardly such that the latching tooth can be advanced over the boss 52 protruding laterally from the cartridge body of the reload cartridge. Desirably, in some embodiments, the arm 154 can be biased laterally inwardly, that is, towards a longitudinal centerline of the reload cover. In some embodiments, the arm 154 can extend in a direction slightly laterally inwardly such that the arm 154 is not joined to the cover surface of the reload cover at a perpendicular angle, but rather, the end of each arm is positioned laterally inwardly of the opposite end of the arm at the cover surface. Thus, in these embodiments when a reload cover 150 is positioned on a reload cartridge 50, each latching tooth 156 securely engages a lateral surface of the reload cartridge and is not readily manually removable.

With continued reference to FIGS. 4-5, the latching tooth 156 can extend laterally inwardly from the end of the arm member of the snap feature 152, and the latching tooth 156 can comprise a latch surface 158 on an upper side or surface thereof and a tapered edge 160 on a lower side thereof. The tapered edge 160 is configured with a transverse extent relative to the generally planar surface of the reload cover such that the tapered edge 160 can engage various features, such as the boss 52 of the reload cartridge 50 during initial assembly of the reload cover 150 to the reload cartridge 50, to flex the arm 154 laterally outwardly. The latch surface 158 can engage a lower, engagement surface of the boss 52 to maintain the reload cover 150 on the reload cartridge. During installation and proper fitting of the reload cartridge 50 and reload cover 150 assembly to a jaw assembly of a surgical stapler, the tapered edge 160 can engage the jaw assembly as further described below and illustrated in FIG. 6 to flex the snap feature 152 laterally outward such that the latch surface 158 disengages the lower, engagement surface of the boss 52 and the reload cover 150 is separated from the reload cartridge 50.

With reference to FIG. 5, an aperture 162 can be formed through the reload cover 150 adjacent each snap feature 152. Thus, in the illustrated embodiment each aperture 162 can be positioned at the distal end of the reload cover at a laterally outward position. As illustrated each snap feature 152 is positioned laterally outwardly of an aperture 162. In certain embodiments, the reload cover can have a generally rectangular profile sized to cover the plurality of rows of staple pockets on the reload cartridge, and the snap features can be disposed on lateral extensions or wings positioned laterally outwardly of the generally rectangular profile. An aperture 162 can be formed in each lateral extension. The aperture 162 can provide a locally flexible portion of the reload cover 150 to allow flexing of the snap feature 152 relative to the body of the reload cover 150. Additionally, the aperture 162 can allow a user to flex the snap features 152 to release the reload cover 150 from the reload cartridge 50 with a hand tool, such as a small screwdriver inserted through the aperture 162. Thus, while assembly of the reload cover 150 with the reload cartridge can be difficult to manually remove to prevent, for example, inadvertent uncovering of staple pockets, in certain instances the reload cover can be removed through the use of a hand tool.

With reference to FIG. 6, a cross-sectional end view of the jaw assembly 30 of an embodiment of surgical stapler is illustrated. As illustrated, the reload cartridge 50 and reload cover 150 have been positioned in the second jaw 32 and partially installed. To position the reload cartridge 50 in the reload support of the second jaw 32, a user initially positions a proximal end of the reload cartridge 50 in the reload support at the proximal end. The user then pivots the distal end of the reload cartridge downwardly onto the distal end of the reload support. The reload support of the second jaw 32 can comprise a channel sized and configured to receive a reload cartridge. The reload support comprises at least one engagement notch 132 or release recess corresponding to the at least one boss 52 and snap feature 152. As the reload cartridge 50 is properly positioned in the reload support such that the boss 52 on the cartridge body seats in the engagement notch 132 of the reload support, the tapered edge 160 of the latching tooth 156 of the snap feature 152 engages a surface of the engagement notch 132. In certain embodiments, the engagement notch 132 can be radiused or tapered to facilitate engagement with the latching tooth 156. Thus, with the reload cartridge 50 properly positioned in the reload support, the snap feature 152 is flexed laterally outwardly such that the latch surface 158 of the latching tooth 156 is disengaged from the boss 52, allowing removal of the reload cover 150. Moreover, as the distal end of the reload cartridge is seated in the reload support, the reload cover 150 is separated from the reload cartridge. FIG. 6 illustrates an initial engagement of the tapered edge 160 with the engagement notch 132 such that further downward movement of the reload cartridge 50 and reload cover 150 relative to the reload support would further flex the snap features 152. Thus, desirably, the tissue contact surface of the reload cartridge 50 can not easily be uncovered for use until the reload cartridge is properly positioned in the reload support of the jaw assembly 30. Failure to properly install reload cartridges within surgical staplers can lead to an incomplete or malformed staple line. Once a reload cartridge is installed to the reload support and the reload cover separated and removed from the reload cartridge, a user can complete a staple firing operation. Then the fired reload cartridge can be removed from the reload support and a second reload cartridge assembly comprising a reload cartridge and a reload cover can be inserted to the reload support as described above for use in a subsequent firing operation.

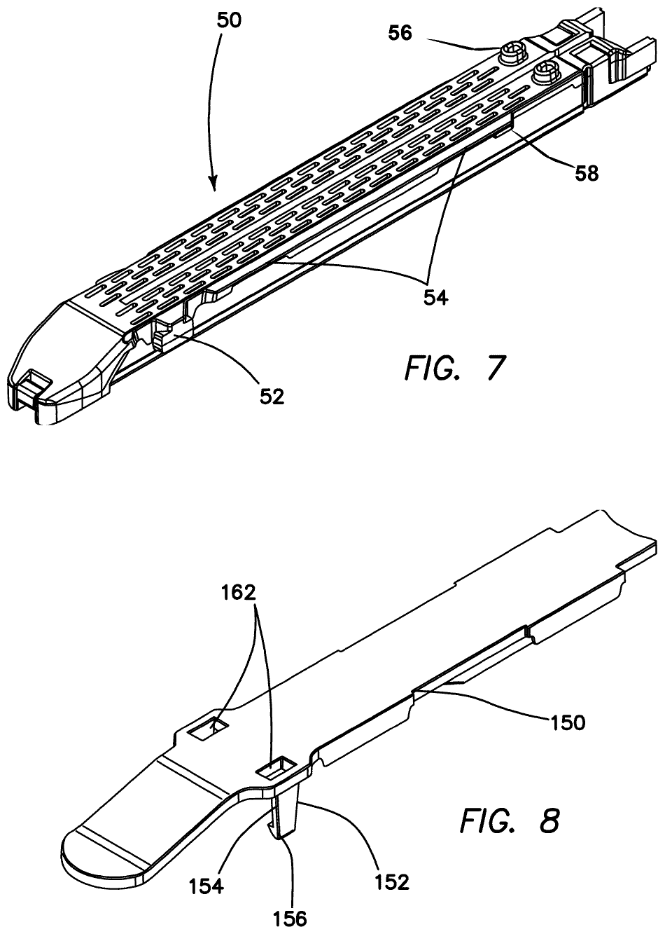

With reference to FIG. 7, a perspective view of the reload cartridge 50 of an embodiment of surgical stapler is illustrated. As illustrated the reload cartridge 50 has a generally elongate profile extending from a proximal end to a distal end. The reload cartridge 50 comprises an upper, tissue contacting surface and a pair of lateral surfaces. The upper surface of the reload cartridge 50 can include a plurality of longitudinally extending rows of staple pockets having a blade channel extending therebetween. In the illustrated embodiment, the upper surface can further comprise at least one seating boss 56 protruding upwardly from the proximal end of the reload cartridge 50. The seating boss 56 can facilitate maintenance of a desired spacing between jaws of the jaw assembly and seating of the reload cartridge when the stapler is closed and fired. As illustrated, the reload cartridge further comprises protruding bosses 52 extending laterally outwardly from the lateral surfaces adjacent a distal end of the reload cartridge 50. The reload cartridge 50 can also comprise at least one notch 54 or recess formed on a laterally protruding rail 58 at a location proximal the bosses 52. In the illustrated embodiment, the reload cartridge is substantially symmetric about the central longitudinal axis and each lateral surface includes a protruding boss 52 and laterally protruding rail 58.

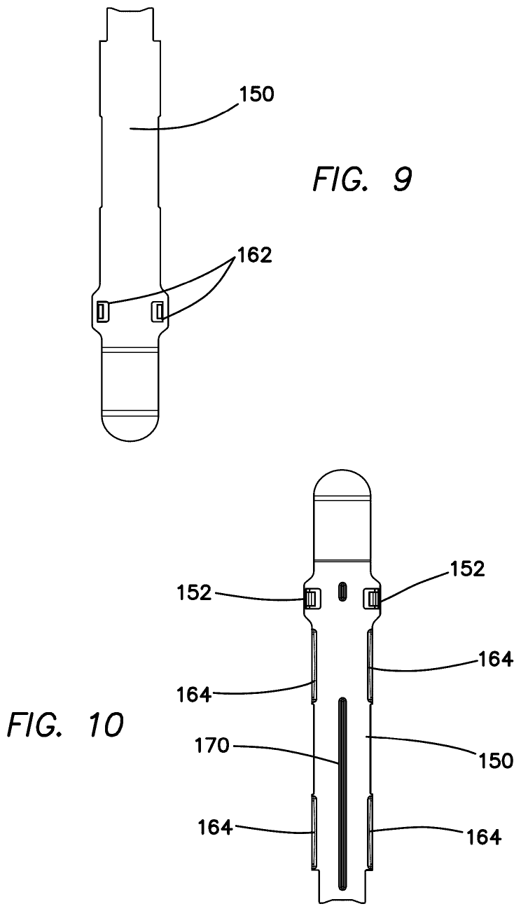

With reference to FIGS. 8-12, various views of an embodiment of reload cover 150 of an embodiment of surgical stapler are illustrated. As illustrated, the reload cover 150 comprises a body having a generally planar configuration with a first surface configured to abut, contact, or be positioned adjacent the upper surface of a reload cartridge. The body of the reload cover can have an elongate configuration with a generally rectangular profile sized to cover the plurality or rows of staple pockets of a reload cover. A proximal end of the body of the reload cover can be sized and configured to contact or abut a seating boss at a proximal end of a reload cartridge. The body further comprises a second surface opposite the first surface. The reload cover comprises a tongue extending distally from the generally planar surface of the body. In the illustrated embodiment, the tongue can extend distally along a plane transverse to the plane defined by the generally planar surface of the body. The tongue can be configured to overlie a distal tapered end of the reload cartridge and extend further distally to a user-graspable distal tip.

With continued reference to FIGS. 8-12, the reload cover 150 comprises at least one snap feature 152 engageable with a boss on a reload cartridge and releasable upon installation in a jaw assembly. As illustrated, the reload cover 150 comprises a pair of snap features 152 extending transversely to the generally planar body adjacent opposite lateral edges at a distal end thereof. In the illustrated embodiments, the snap features 152 each include an arm 154 having a latching tooth 156 with a latch surface 158 and a tapered edge 160. The reload cover can further comprise apertures 162 formed adjacent the snap features 152.

With reference to FIGS. 10-12, in certain embodiments, the reload cover can comprise one or more guide tabs or retention tabs 164 positioned along lateral edges of the body of the reload cover 150 proximal the snap features 152. The retention tabs 164 can be positioned to engage the notches 54 on the laterally extending rails of the reload cartridge (FIG. 7) to facilitate positioning and retention of the reload cover 150 on the reload cartridge 50. In certain embodiments, the guide tabs or retention tabs can each comprise a tip member such as a bulbous edge or a laterally inwardly extending flange configured to engage the recesses or notches 54 on the rails of the reload cartridge in a snap fit coupling. In the illustrated embodiment, the reload cover comprises a pair of distal retention tabs 164 positioned adjacent to and proximal to the snap features 152 and a pair of proximal retention tabs 164 positioned adjacent the proximal end of the reload cover 150 and proximal the distal retention tabs. In other embodiments, the reload cover can include more or fewer retention tabs such as, for example one pair of retention tabs or three pairs of retention tabs.

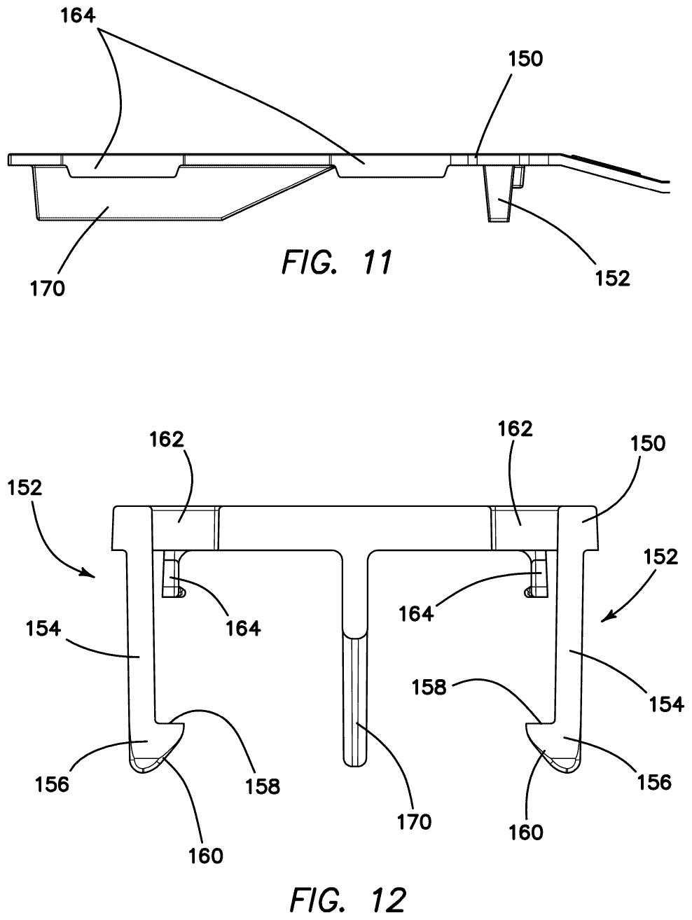

With reference to FIGS. 11-12, in certain embodiments, the reload cover 150 can comprise a fin 170 or alignment fin extending downwardly from the first surface of the reload cover 150 and positioned to be disposed in the blade channel of the reload cartridge. In the illustrated embodiment, the fin 170 has an elongate configuration, such that it can extend longitudinally within the blade channel to maintain a longitudinally aligned orientation of the reload cover with respect to the reload cartridge. In other embodiments, the fin 170 can have a reduced length and can comprise a post or arm positionable in the blade channel of the reload cartridge. The fin 170 can be positioned such that a proximal end of the fin 170 prevents distal movement of a staple deployment mechanism such as a sled or slider within the reload cartridge when the reload cover 150 is positioned on the reload cartridge 50. Thus, desirably, the fin 170 of the reload cover can prevent distal migration of a staple deployment mechanism prior to installation of the reload cartridge in the jaw assembly. If the staple deployment mechanism or slider were to distally translate prior to installation of the reload cartridge 50, in certain instances even an unfired cartridge would not defeat various fired reload lockout mechanisms which can be included in a stapling assembly. In certain embodiments, a fired reload lockout mechanism can comprise a lockout lever positioned adjacent a firing member of the stapler, and the fin 170 can maintain the staple deployment mechanism in a position to engage the lockout lever. Thus, desirably, the reload cover 150 can prevent inadvertent operation of a fired reload lockout mechanism of a stapler by an unfired reload cartridge.

Although this application discloses certain preferred embodiments and examples, it will be understood by those skilled in the art that the present inventions extend beyond the specifically disclosed embodiments to other alternative embodiments and/or uses of the invention and obvious modifications and equivalents thereof. Further, the various features of these inventions can be used alone, or in combination with other features of these inventions other than as expressly described above. Thus, it is intended that the scope of the present inventions herein disclosed should not be limited by the particular disclosed embodiments described above, but should be determined only by a fair reading of the claims which follow.

What is claimed is:

1. A surgical stapling device comprising:
an elongate shaft;
a reload cartridge having a proximal end and a distal end and extending longitudinally from the proximal end to the distal end, the reload cartridge comprising at least one protruding boss extending laterally outwardly therefrom at the distal end;
a jaw assembly, the jaw assembly comprising a reload support configured to receive the reload cartridge; and
a reload cover having a proximal end and a distal end and extending longitudinally from the proximal end to the distal end, the reload cover releasably positionable on the reload cartridge, the reload cover comprising at least one latch arm extending therefrom at the distal end and a corresponding at least one aperture adjacent the at least one latch arm, each latch arm engageable with a corresponding protruding boss;
wherein the reload cover is removable from the reload cartridge upon one of: installation of the reload cartridge and reload cover to the reload support to disengage the at least one latch arm from the at least one protruding boss, and insertion of a tool through the at least one aperture to selectively disengage the at least one latch arm from the at least one protruding boss.

2. The surgical stapling device of claim 1, wherein each of the at least one latch arms is biased laterally inwardly.

3. The surgical stapling device of claim 1, wherein each of the at least one latch arms extends in a direction laterally inwardly.

4. The surgical stapling device of claim 1, wherein the reload support comprises at least one engagement notch formed therein positioned to engage the at least one latch arm when the reload cartridge and the reload cover are installed in the reload support.

5. The surgical stapling device of claim 4, wherein the at least one latch arm comprises an arm member extending from the reload cover to an end, and a latch tooth extending laterally inwardly from the arm member at the end.

6. The surgical stapling device of claim 5, wherein the latch tooth has a release surface positioned to engage the engagement notch to displace the latch tooth laterally outwardly upon installation of the reload cartridge to the reload support.

7. The surgical stapling device of claim 6, wherein the latch tooth has a lower side and wherein the release surface comprises a tapered edge on the lower side.

8. The surgical stapling device of claim 6, wherein the latch tooth has an upper side and wherein the latch tooth comprises a latch surface on the upper side thereof.

9. The surgical stapling device of claim 8, wherein the at least one protruding boss has a lower surface, and wherein the latch surface of the latch tooth engages the lower surface of the protruding boss when the reload cover is positioned on the reload cartridge.

10. The surgical stapling device of claim 1, wherein the reload cartridge comprises a blade channel and wherein the reload cover comprises a fin positionable within the blade channel when the reload cover is positioned on the reload cartridge.

11. The surgical stapling device of claim 1, wherein each of the at least one latch arm is positioned laterally outwardly of a corresponding one of the at least one aperture.

12. The surgical stapling device of claim 11, wherein the reload cover comprises a rectangular profile and further comprises at least one lateral extension positioned laterally outwardly of the rectangular profile and wherein each of the at least one latch arm extends from a corresponding one of the at least one lateral extension.

13. The surgical stapling device of claim 12, wherein each of the at least one apertures is formed in a corresponding one of the at least one lateral extension.

14. A reload cartridge assembly for a surgical stapler, the reload cartridge assembly comprising:
a cartridge body having a generally elongate profile extending from a proximal end to a distal end, and the cartridge body comprising:
an upper surface having a plurality of rows of staple pockets formed therein and a blade channel extending between two adjacent rows of the plurality of rows of staple pockets; and
a pair of lateral surfaces, the pair of lateral surfaces each comprising a protruding boss extending laterally outwardly at the distal end of the cartridge body; and
a reload cover removably positionable on the upper surface, the reload cover having a generally rectangular profile and a generally elongate profile extending from
a proximal end to a distal end; the reload cover comprising:
a pair of lateral extensions positioned laterally outwardly of the rectangular profile at the distal end of the reload cover;
a pair of latch arms each extending downwardly and laterally inwardly from a corresponding one of the pair of lateral extensions; and
a pair of apertures through the reload cover, each aperture positioned adjacent to a corresponding one of the pair of latch arms;
wherein each of the latch arms is engageable with a corresponding protruding boss to retain the reload cover to the upper surface of the cartridge body.

15. The reload cartridge assembly of claim 14, wherein each latch arm of the pair of latch arms comprises an arm member extending from the reload cover to an end, and a latch tooth extending laterally inwardly from the arm member at the end.

16. The reload cartridge assembly of claim 15, wherein the latch tooth has a release surface configured to displace the latch tooth laterally outwardly upon installation of the reload cartridge to the surgical stapler.

17. The reload cartridge assembly of claim 16, wherein the latch tooth has a lower side and wherein the release surface comprises a tapered edge on the lower side.

18. The reload cartridge assembly of claim 16, wherein the latch tooth has an upper side and wherein the latch tooth comprises a latch surface on the upper side thereof.

19. The reload cartridge assembly of claim 18, wherein the protruding boss has a lower surface, and wherein the latch surface of the latch tooth engages the lower surface of the protruding boss when the reload cover is positioned on the reload cartridge.

20. The reload cartridge assembly of claim 14, wherein the reload cover comprises a fin positionable within the blade channel when the reload cover is positioned on the reload cartridge.
